(19) United States
(12) Patent Application Publication
(10) Pub. No.: US 2004/0260146 A1
(43) Pub. Date: Dec. 23, 2004

(54) Oscillating intercourse simulator

(76) Inventor: Guanlong Cao, Malden, MA (US)

Correspondence Address: GUANLONG CAO, Legend Photography, 36 Winter St., Malden, MA, 02148, US

(21) Appl. No.: 10/462,960

(22) Filed: Jun. 18, 2003

(51) Int. Cl. A61F 5/00

(52) U.S. Cl. 600/38

(57) ABSTRACT

Oscillating Intercourse Simulator is a sex device simulating intercourse. The artificial penis is propelled by the oscillating pendulum. The pendulum is driven by the crankshaft with the electric motor. To adjust the oscillating amplitude, slide the crankshaft up, or down, along the pendulum. When the crankshaft slides up, the oscillating amplitude increases; when it slides down, the amplitude decreases.

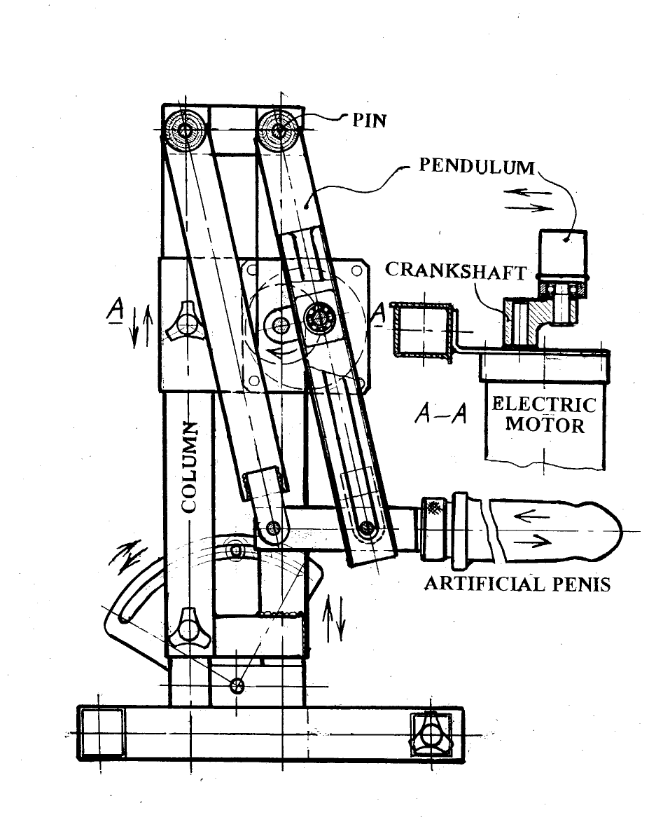

### BRIEF SUMMARY OF THE INVENTION

Oscillating Intercourse Simulator is a sex device simulating intercourse. The artificial penis is propelled by the oscillating pendulum. The pendulum is driven by the crankshaft with the electric motor. To adjust the oscillating amplitude, slide the crankshaft up, or down, along the pendulum. When the crankshaft slides up, the oscillating amplitude increases; when it slides down, the amplitude decreases.

### BRIEF DESCRIPTION OF THE DRAWING

The artificial penis is mounted on the end of, and being propelled by, the oscillating pendulum. The pendulum is driven by the crankshaft with the electric motor. To adjust the oscillating amplitude, slide the crankshaft up, or down, along the pendulum. When the crankshaft slides up, the oscillating amplitude increases; when it slides down, the amplitude decreases.

Oscillating Intercourse Simulator is a sex device simulating intercourse. Its claims are:

1. The structure in which the artificial penis is propelled by the oscillating pendulum.

2. The structure of sliding the crankshaft up, or down, along the pendulum to adjust the amplitude of the oscillating.
